(19) United States
(12) Patent Application Publication
(10) Pub. No.: US 2004/0120972 A1
(43) Pub. Date: Jun. 24, 2004

(54) Equine herpes virus temperature sensitive mutant and live vaccine thereof

(76) Inventor: Jay R. Patel, Caldecote (GB)

Correspondence Address: INTERVET INC, 405 STATE STREET, PO BOX 318, MILLSBORO, DE, 19966, US

(21) Appl. No.: 10/729,078

(22) Filed: Dec. 4, 2003

Related U.S. Application Data
(62) Division of application No. 10/070,285, filed on Mar. 5, 2002.
Application No. 10/070,285 is a 371 of international application No. PCT/EP00/08944, filed on Sep. 11, 2000.

(30) Foreign Application Priority Data
Sep. 10, 1999 (EP) 99202933.0

(51) Int. Cl. A61K 39/12; C12N 7/00

(52) U.S. Cl. 424/204.1; 435/235.1

(57) ABSTRACT

The present invention relates to an equine abortion virus (EHV-1) mutant which is temperature sensitive at the body temperature of the host animal, more specifically at a temperature of 38.5° C. or higher. The temperature sensitive mutant can be used in vaccination to protect susceptible animals against EHV-1 infection. The invention furthermore relates to live vaccines derived from said mutant.

The present invention relates to an equine abortion virus mutant, a process for the preparation of said mutant, use of said mutant and live vaccines derived from said mutant.

Equine abortion virus (EHV-1), a herpes virus, is a major equine pathogen responsible for viral-induced abortion. neurological disease such as paresis, infections of the upper respiratory tract, and neonatal foal disease (NFD). NFD results from close to term transplacental infection of fetuses, which are born weak with severe respiratory disease and some with jaundice due to liver infection by EHV-1. These animals usually die within a few days after birth. Equine rhinopneumonitis virus (EHV-4) is the major cause of acute respiratory tract disease (“rhinopneumonitis”) and infects most horses during their first two years of life. Rhinopneumonitis is characterized by fever, anorexia, and profuse serous nasal discharge that later becomes mucopurulent. On rare occasions EHV4 infection causes abortion in pregnant mares. Furthermore EHV1 and EHV4 establish persistent, lifelong latent infections. Upon reactivation the viruses cause recurrent disease, accompanied by virus shedding and transmission to other animals.

Control of equine herpes virus infection and their diseases remain inadequate, in particular against EHV1 mediated abortions, paresis and neonatal foal disease resulting from close to term transplacental infection of foetus. Although inactivated as well as modified live vaccines are available, neither vaccine appears to block infection sufficiently, nor do they prevent the establishment of latency by wild-type virus. Hence there is a great need for safe vaccines with improved protection against field infections of these viruses, particularly against infections caused by EHV1.

The present invention provides for such vaccines.

In a first aspect the present invention provides for an EHV-1 Ts mutant as deposited at the European Collection of Animal Cell Culture (ECACC), Salisbury, Wiltshire SP4 0JG, UK on 10 Jun. 1999 under accession number V99061001, and progeny thereof.

The EHV-1 Ts mutants according to the invention are furthermore phenotypically characterized in that

they form small plaques when grown on several horse cell lines,

they have lost their ability to grow on rabbit kidney cells, in particular RK13 cells, and

they are limited in their ability to cause viraemia (that is, they are able to;

The EHV-1 Ts mutants according to the invention have the advantage that replication is restricted to the upper respiratory tract of conventional equidae with no or limited ensuing viraemia. The Ts mutants are safe for pregnant mares while giving rise to significant immune stimulation following growth in the upper respiratory tract. The Ts mutants are not readily back-passaged form animal to animal thus limited in their potential for transmission and reversion.

For the purpose of this invention “progeny” is defined to include also all strains obtained by further serial passage of the deposited EHV-1 Ts mutant.

For the purpose of this invention, a temperature sensitive mutant is defined as a mutant virus which has an impaired growth at or above a certain temperature at which the wild type has a normal growth. The EHV-1 Ts mutants according to the present invention are characterized in that they are temperature sensitive at the body temperature of the host animal. The EHV-1 Ts mutants of the present invention do not replicate above a temperature of 38.5 to 39.0° C. Preferably the EHV-1 Ts mutants according to the invention do not replicate at a temperature of 38.5° C.

For the purpose of this invention, small plaques are defined as plaques that are at least half to one third the size of the plaques formed by the wild-type parent strain in equine cells.

For the purpose of this invention the “limited abilty to cause viraemia” is defined as the ability to cause no or low grade (that is, just detectable) vireamia for 1 to 3 or 4 days in some animals with respect to the ability of the parent strain to cause viraemia.

Temperature sensitive EHV-1 mutants according to the invention can be obtained by treatment of infected bovine, equine or other permissive cell cultures at 34° C. with non-toxic concentrations of a mutagens such as 5-bromo-2-deoxy uridine, azacytidine and the like during viral replication in vitro, followed by biological cloning of progeny virus from said treated cultures in bovine or equine or other permissive cell lines.

The favorable properties of the Ts-mutants according to the invention makes them very suitable for use in the preparation of a vaccine. Thus, in a second aspect the present invention provides for a composition, in particular a vaccine composition, comprising an EHV-1 Ts-mutant according to the invention, and a pharmaceutically acceptable carrier or vehicle. More specifically, a (vaccine) composition according to the invention comprises the EHV1 Ts-mutant deposited at the ECACC, Salisbury, UK having accession number V99061001 and/or progeny thereof. Pharmaceutical acceptable carriers or vehicles that are suitable for use in a vaccine according to the invention are sterile water, saline, aqueous buffers such as PBS and the like. In addition a vaccine according to the invention may comprise other additives such as adjuvants, stabilizers, anti-oxidants and others.

The vaccine compositions according to the invention are safe and can be used to protect the equidae clinically and virologically against infections with EHV-1 and to protect against virus-induced abortions and paresis. In addition the vaccine according to the invention was found to stop trans-placental infection, thus protecting the newborn foal from the effects of neonatal foal disease. The vaccine composition according to the present invention can be administered not only to horses but also to other animals that are susceptible to EHV-1 infection such as donkeys, zebra's and the like. Cattle which have been reported to be susceptible to EHV-1 and EHV-4 infection can also be treated with the vaccine according to the invention.

It was furthermore surprisingly found that vaccines comprising an EHV-1 Ts-mutant according to the invention not only protect against EHV-1 infections but also against the disease and the associated virus shedding following EHV-4 infection. Thus such a vaccine can be useful to obtain cross-protection in the vaccinated equidae. Said vaccines give rise to improved protection thus effectively blocking infection with wild-type viruses.

Vaccine compositions according to the invention can be prepared following standard procedures. A vaccine according to the invention preferably is a live vaccine. For the preparation of the live vaccine, the seed virus of the EHV-Ts mutant can be grown on a cell culture, such as primary or secondary bovine kidney or equine cells. The viruses thus grown can be harvested by collecting the tissue cell culture fluids and/or cells. Optionally, during harvesting the yield of the viruses can be promoted by techniques that improve the liberation of the infective particles from the growth substrate, e.g. sonication. The live vaccine may be prepared in the form of a suspension or may be lyophilized.

Pharmaceutical acceptable carriers that are suitable for use in a vaccine according to the invention are sterile water, saline, aqueous buffers such as PBS and the like. In addition a vaccine according to the invention may comprise other additives such as adjuvants, stabilizers, anti-oxidants and others.

Suitable stabilizers are for example carbohydrates including sorbitol, mannitol, starch, sucrose, dextran and glucose, proteins and degradation products thereof including but not limited to albumin and casein, protein-containing agents such as bovine serum or skimmed milk, and buffers including but not limited to alkali metal phosphates. In lyophilized vaccine compositions it is preferable to add one or more stabilizers.

Suitable adjuvants include but are not limited to aluminum hydroxide, phosphate or oxide, amphigen, tocopherols, monophosphenyl lipid A, muramyl dipeptide, oil emulsions, glucans, carbomers, block-copolymers, cytokines and saponins such as Quil A. The amount of adjuvant added depends on the nature of the adjuvant itself.

EHV-1 Ts mutants according to the invention are preferably administered to conventional, seronegative animals varying in ages from a few days to several years, including those in-foal. The vaccine can be administered to the animals via non-parenterally administration routes, including but not limited to intradermal, oral, spraying, aerosol, intra-ocular, and intranasal administration. Alternatively, the vaccine can be administered via parenteral administration routes. Preferably the vaccine is administered intradermally or intranasally.

In general the EHV-1 Ts mutant virus is administered in an amount that is effective to induce protection against EHV-1 infection. The dose generally will depend on the route of administration, the time of administration, as well as age, health and diet of the animal to be vaccinated. The virus can be administered in an amount between 10² and 10⁹ pfu/dose per animal, preferably between 10³ and 10⁵ pfu/dose and more preferably at 10⁴ pfu/dose per animal.

The vaccines according to the invention also may be given simultaneously or concomitantly with other live or inactivated vaccines. These additional vaccines can be administered non-parenterally or parenterally. Preferably the additional vaccines are recommended for parenteral administration.

### EXAMPLES

### 1. Isolation and Characterization of a Temperature Sensitive EHV-1 Mutant Strain TS C147

Just confluent, day-old 75 cm² monolayers of equine dermal (ED) cells were infected at m.o.i. of 0.001 with EHV-1. Inoculum (2.0 ml) was adsorbed (1 hour, 37° C.), removed and monolayers were washed with PBS and then re-fed with tissue culture medium (25 ml) containing 40 μg/ml of 5-bromo-2-deoxy uridine and incubated at 34° C. At maximum CPE (7 days post inoculation), the culture was harvested (frozen at 40° C. and then thawed at 37° C.), dialyzed overnight at 4° C. against PBS, titrated for EHV-1 infectivity in ED cells at 37° C. and subsequently cloned at 34° C. in ED cells grown in 96-well microtitration plates. Wells with single EHV-1 focus were identified, allowed to grow to maximum CPE and then a small (20 μl out of 200 μl total) sample used for phenotyping at permissive (34° C.) and restrictive (39° C.) temperatures using ED cells. Temperature sensitive clones were further passaged in Bovine Kidney cells, strain JCK (Jay's Calf Kidney—Intervet's strain) to produce the master and working seeds.

### 2. Temperature Sensitive Phenotye of EHV-1 Strain TS C147

EHV-1 TS C147 strain at Master Seed Virus (MSV)+1° level was titrated in parallel in Bovine Embryo Lung (strain BEL₂₆—Intervet's strain), Bovine Kidney (strain Jay's Calf Kidney, JCK—Intervet's strain), Equine Dermal (ED) cells, Equine Dermal Clone W48 C10 (ED W48 C10—Intervet's strain), and Equine Dermal Clone W7 C5 (ED W7 C5—Intervet's strain) at 37° C. and 38.5° C. Virus at MSV+1° passage level failed to grow at 38.5° C. Results are given in Table 1.

### 3. EHV-1 Strain TS C147 has EHV-4 like Characteristics

A parameter for the differentiation between EHV-1 and EHV-4 is their ability to replicate in rabbit kidney (RK13) cells. EHV-1 strains replicate well in RK13 cells but the cells are refractory to EHV-4 strains. EHV-1 strain TS C147 at MSV+1° level, its wild type parent EHV-1 strain, EHV-1 strain deficient in immediate early gene (EHV-1 IE), pathogenic EHV-1 strain CHLi and a field EHV-4 isolate were titrated in parallel at 37° C. in RK13 cells and Equine dermal (ED) cells. Results given in Table 2 show that the 4 EHV-1 strains, including strain TS C147 and EHV-4 strain replicated in ED cells but EHV-1 strain TS C147 and EHV-4 strain did not grow in RK13 cells.

TABLE 1
Relative titers of EHV-1 strain TS C147 at 37° and
38.5° in various bovine and equine cell strains

| Titers (log₁₀ TCID₅₀/ml) at | | |
|---|---|---|
| 37° C. and 38.5° C. in various | | |
| bovine and equine cell types | | Virus Passage level TSC147 MSV + 1° |
| BEL₂₆: | 37° C. | 5.2 |
| | 38.5° C. | <1.1 |
| JCK: | 37° C. | 5.4 |
| | 38.5° C. | <1.1 |
| ED: | 37° C. | 5.4 |
| | 38.5° C. | <1.1 |
| ED W48 C10: | 37° C. | 5.7 |
| | 38.5° C. | <1.1 |
| ED W7 C5: | 37° C. | 5.7 |
| | 38.5° C. | <1.1 |

a) Titers after 5 days' incubation; Titters given as <1.1 log₁₀ TCID₅₀/ml represent no EHV-1 foci detected in 4 × 200 μl of the lowest (10⁻¹) dilution of the virus tested in the titration

TABLE 2
Ability of rabbit kidney cells to support replication
of EHV-1 strain TS C147

Relative titer
(log₁₀ TCID₅₀/ml)
at 37° C. in RK13 & ED cells

| Virus | RK13 cells | ED cells |
|---|---|---|
| EHV-1 TS C147 MSV + 1° | <1.1^{a} | 5.7 |
| EHV-1 040 | 5.7 | 5.7 |
| EHV-1 IE | 6.0 | 6.2 |
| EHV-1 CHLi | 5.7 | 6.0 |
| EHV-4 | <1.1 | 3.7 |

^{a}= Titers given as <1.1 log₁₀ TCID₅₀/ml represent no EHV-1 CPE detected in 4 × 200 μl of the lowest dilution (10⁻¹) in the titration.

### 4. Clinical and Virological Protection of Conventional Ponies Against Infections by EHV-1 and EHV-4

Of 29 conventional ponies with low or no EHV-1 neutralizing (VN) antibody, 15 were vaccinated intranasally (IN) with a dose of 5.3 log₁₀ TCID₅₀ of strain EHV-1 TS C147 while 14 ponies were left unvaccinated to serve as unvaccinated control. About a month following a single IN vaccination, 8 vaccinated and 8 unvaccinated (control) ponies were challenged IN with a field strain of EHV-1 while a group of 7 EHV-1 vaccinated and 6 control animals were challenged IN with a recent field isolate of EHV-4. Following vaccination and challenge, animals were monitored for clinical reactions, virus shedding in nasal mucus, infected leukocytes (viraemia) and EHV-1 neutralizing antibody.

Vaccine virus grew to low titers (nasal mucus peak titers 1.5 to 3.0 log₁₀ TCID₅₀/ml) for 1 to 8 days in 11/15 ponies and also resulted in low-grade (just detectable) leukocyte viraemia for 1 to 4 days in 7 of 15 animals. However all 15 ponies seroconverted. In contrast no EHV-1 was recovered from the nasal mucus or the blood of 14 control ponies monitored daily for 10 or 14 days respectively and the animals remained seronegative to EHV-1 until after challenge infection. A similar level of pyrexia was seen in 10 animals in each of the two (vaccinated and control) groups. These findings are summarized in Table 3.

Following intranasal EHV-1 challenge, there was a significant reduction in virus excreted in nasal mucus by the vaccinated ponies relative to that recovered from the control animals. Similarly a single vaccination prevented leukocyte viraemia in 7 of 8 ponies while one pony was just virus positive for 1 day. In contrast, however all 8 unvaccinated ponies became viraemic, 7 for 3 to 4 days and 1 for 1 day. All 8 control ponies became moderately to highly febrile for 1 to 6 days but all 8 vaccinated animals remained normal. None of the 8 vaccinated animals responded anamnestically to the challenge infection while all 8 control animals responded with a significant EHV-1 neutralizing antibody. These findings are summarized in Table 4.

Following intranasal EHV-4 challenge, virus was recovered from the nasal mucus of one of 7 vaccinated ponies on one occasion but all 6 control ponies excreted virus at a significantly higher titer for 2 to 3 days, with one exception. None of the 7 EHV-1 vaccinated ponies became viraemic in contrast to 3 of 6 control ponies for 1-3 days. EHV-4 challenge infection resulted in pyrexia in 3 of 6 control animals for 2 to 3 days but none of the 7 vaccinated ponies were affected. There was a slight (15 to 20 expirations/minute) increase in respiration rate in 4 of 7 vaccinated and 5 of 6 control animals for 1 to 3 and 2 to 6 days respectively. These findings are summarized in Table 5.

TABLE 3
Results after vaccination
Result - No +ve/No Total (Peak activity range & duration)

| Parameter | Vaccinated | Control |
|---|---|---|
| Virus shedding in mucus | 11/15 (1.5-3.0 log₁₀ TCID₅₀/ml, | 0/14 |
| | 1-8 days) | |
| Leukocyte viraemia | 7/15 (low grade, 1-4 days) | 0/14 |
| Seroconversion | 15/15 | 0/14 |
| Pyrexia (= 38.5° C.) | 10/15 (38.5-39.3, 1-3 days) | 10/14 |
| (Between days 1-10) | | (38.5-38.8, |
| | | 1-3 days) |

TABLE 4
After EHV-1 challenge
Result - No +ve/No Total (Peak activity range & duration)

| Parameter | Vaccinated | Control |
|---|---|---|
| Virus shedding in | 5/8 (1.5 log₁₀/ml, | 8/8 (2.2-3.4 log₁₀ |
| mucus | 1-2 days) | TCID₅₀/ml, |
| | | 4-6 days) |
| Leukocyte viraemia | 1/8 (low grade, | 8/8 (3-4 days; |
| | 1 day) | 1 day for 1 |
| | | animal) |
| EHV-1 (VN) antibody | 0/8 (=4-fold rise) | 8/8 |
| rise) | | |
| Pyrexia | 0/8 | 8/8 (38.9-41.0, |
| | | 1-6 days) |
| Respiration | 3/8 (15-20 expirations/ | 2/8 (15-20 |
| | min, 1 day) | expirations/min |
| | | 1 day) |

TABLE 5
Results after EHV-4 challenge
Result - No +ve/No Total (Peak activity range & duration)

| Parameter | Vaccinated | Control |
|---|---|---|
| Virus shedding | 1/7 (1.5 log₁₀/ml, | 6/6 (1.5-3.7 log₁₀ |
| in mucus | 1 day) | TCID₅₀/ml, |
| | | 1-3 days) |
| Leukocyte viraemia | 0/7 | 3/6 (1-3 days) |
| EHV-1 (VN) | 1/7 (=4-fold rise) | 5/6 |
| antibody rise) | | |
| Pyrexia | 0/7 | 3/6 (38.6-38.8, |
| | | 2-3 days) |
| Respiration | 4/7 (15-20 | 5/6 (15-20 |
| | expirations/min, | expirations/min |
| | 1-3 days) | 2-6 days) |

### 5. Protection of Equidae Against Paresis and Abortions due to EHV-1 Infection

Of 12 pregnant mares with low or no EHV-1 neutralizing (VN) antibody, 6 were vaccinated intranasally (IN) at about 6 months of gestation and then all 12 mares challenged IN with a pathogenic strain of EHV-1 at the critical stage of gestation for EHV-1 abortions namely about 9 months of gestation. Following vaccination and challenge, animals were monitored for clinical reactions, virus shedding in nasal mucus, infected leukocytes (viraemia) and EHV-1 neutralizing antibody.

Although no vaccine virus was recovered from nasal mucus from any of 6 vaccinated mares, low grade, transient (1 to 3 days) viraemia was detected in 5 of 6 mares and all 6 animals seroconverted with significant VN antibody to EHV-1. None of the 6 control mares, monitored in parallel to vaccinated animals for 10 to 14 days, yielded EHV-1 from nasal mucus or leukocytes but 1 of 6 animals seroconverted some 2½ months later. These findings are summarised in Table 6.

Following challenge, there was a significant (2 out of 6 compared to 5 of 6 and 1.5 to 1.7 log₁₀ TCID₅₀/ml for 1-2 days compared to 2.4 to 3.7 log₁₀ TCID₅₀/ml for 1-6 days) reduction in virus excreted in nasal mucus by the vaccinated mares. Similarly none of 6 vaccinated mares became viraemic in contrast to 5 of 6 unvaccinated control mares. In the control group 5 of 6 mares became febrile for 1 to 5 days, 3 also developed paresis accompanied by severe jaundice and disintegration of the cervical plug in 2 mares with signs of foetal ejection. One of the two animals died while the 2^{nd} had to be euthanased in extremis. Both animals carried dead foals. Three further mares aborted. Foetal tissues from all 5 foetuses were EHV-1 positive. In contrast however all 6 vaccinated mares foaled normally. The only clinical reaction observed in vaccinated mares was transient (1 day) pyrexia in one of 6 mares. The control mare which foaled normally had in fact seroconverted just prior to challenge. These findings are summarised in Table 7.

TABLE 6
Results after vaccination
Result - No +ve/No Total (Peak activity range & duration)

| Parameter | Vaccinated (group 1) | Control (group 2)^{a} |
|---|---|---|
| Virus shedding in mucus | 0/6 | Not monitored^{a} |
| Leukocyte viraemia | 5/6 (low, 1-3 days) | 0/6 |
| Pyrexia | 1/6 (1 day) | Not monitored^{a} |
| Seroconversion | 6/6 (Month onwards after | 1*/6 (About 3 |
| | vaccination) | months after |
| | | vaccination) |

^{a}= Not monitored because animals kept in isolation away from the vaccinated group

TABLE 7
Results after challenge
Result - No +ve/No Total (Peak activity range & duration)

| | Vaccinated | |
|---|---|---|
| Parameter | (group 1) | Control (group 2) |
| Virus shedding | 2/6 | 5/6 (2.4-3.7 log₁₀ TCID₅₀/ml, 1-6 |
| in mucus | (1.5-1.7 log₁₀ | days) |
| | TCID₅₀/ml, | |
| | 1-2 days) | |
| Leukocyte | 0/6 | 5/6 |
| viraemia | | |
| EHV-1 VN | 0/6 | 5/6† |
| antibody rise | | |
| Pyrexia | 1/6 (1 day) | 5/6 (1-5 days) |
| Paresis | 0/6 | 3/6 (Terminal in 2 mares) |
| Jaundice | 0/6 | 2/6 |
| Death | 0/6 | 2/6 (1 died, 1 euthanased in extremis |
| | | with severe paralysis jaundice and |
| | | rapid decrease in body temperature |
| Abortion | 0/6 | 5/6 |

†Control mare was seronegative in 3-monthly bleeds after vaccination of group 1 mares but seroconverted prior to challenge.

### 6. Safety of EHV-1 TS C147 in Pregnant Mares

Four mares at about 9 months of gestation (critical stage for EHV-1 abortions) were inoculated by the natural route with 10 times the protective dose and monitored for abortions. Results given in Table 8 show that all 4 mares seroconverted to EHV-1, one of 4 mares became transiently viraemic but foaled normally. Three of 4 foals were EHV-1 VN antibody negative in blood samples collected before suckling the respective dam while one foal was VN antibody positive due to colostrum intake (born between monitoring intervals in the early hours). These results are summarised in Table 8.

TABLE 8
An overdose safety for pregnant mares at th critical stage of gestation
for EHV-1 abortions

| | Shedding | | | | | |
|---|---|---|---|---|---|---|
| | in | | | | | |
| Mare | nasal | | VN antibody to EHV-1 | | Foaling & | |
| No | mucus | Viraemia | At dosing & 3 wks later | | antibody^{a} | |
| 13 | c | −ve | <2.0 | 3.5 | Normal | <2.0 |
| 14A | c | +ve | <2.0 | 6.0 | Normal | 4.0^{b} |
| | | (3 days) | | | | |
| 15 | c | −ve | <2.0 | 5.0 | Normal | <2.0 |
| 16 | c | −ve | <2.0 | 6.0 | Normal | <2.0 |

^{a}EHV-1 neutralizing antibody at birth
^{b}Born between monitoring intervals in early hours and the foal was bled at least 3 hours after birth
^{c}Pending, ie to be done

### 7. No Transmission of EHV-1 TS C147 Between Target Species

A back-passage study was performed in EHV-naïve (all types) weaned foals (specific pathogen free, SPF foals).

Two SPF foals were inoculated intranasally (IN) with 10 times protective dose of EHV-1 strain TS C147 at Master Seed Virus+1° passage level and virus positive nasal mucus collected over several days used to similarly infect a further pair of SPF foals. After IN inoculation, foals were monitored for (i) virus shedding in nasal mucus, (ii) clinical reactions and (iii) seroconversion to EHV-1.

Foals given EHV-1 strain TS C147 at MSV+1° level excreted virus in nasal mucus and seroconverted. However, a pool of virus positive nasal mucus samples failed to infect a further pair of EHV-naïve foals as judged from the failure to recover EHV-1 from their nasal mucus and the absence of seroconversion to EHV-1. The results were confirmed by repeating the study with a further 4 SPF foals, 2 inoculated with MSV+1° followed by a further 2 given virus positive nasal mucus from the first two foals. Results are summarised in Tables 9 and 10.

TABLE 9
Backpassage of EHV-1 strain TS C147 in EHV-naïve foals

| | |
|---|---|
| Parameter | Result (+/−, range & duration) |

(i) PASSAGE ONE: Foals 1 & 2 inoculated intranasally with
EHV-1 TS C147 (10x protective dose) at MSV + 1° level.

| | |
|---|---|
| Virus shedding in nasal mucus | 2/2 (1.7-5.0 log₁₀ TCID₅₀/ml, 5-6 days) |
| Seroconversion to EHV-1 | 2/2 (bleed 2 weeks after inoculation & by |
| | CF test) |

(ii) PASSAGE TWO: Foals 5 & 6 inoculated intranasally
with virus positive nasal mucus from foals 1 & 2

| | |
|---|---|
| Virus shedding in nasal mucus | 0/2 |
| Seroconversion | 0/2 |

TABLE 10
Backpassage of EHV-1 strain TS C147 in EHV-naïve foals

| | |
|---|---|
| Parameter | Result (+/−, range & duration) |

(i) PASSAGE ONE: Foals 7 & 8 inoculated intranasally with
EHV-1 TS C147 (10x protective dose) at MSV + 1° level.

| | |
|---|---|
| Virus shedding in nasal mucus | 2/2 (1.5-3.7 log₁₀ TCID₅₀/ml, 4-8 days) |
| Seroconversion to EHV-1 | 2/2 (bleed 2 weeks after inoculation & by |
| | CF test) |

(ii) PASSAGE TWO: Foals 9 & 10 inoculated intranasally
with virus positive nasal mucus from foals 7 & 8

| | |
|---|---|
| Virus shedding in nasal mucus | 0/2 |
| Seroconversion to EHV-1 | 0/2 |

1. A temperature-sensitive (Ts) mutant of Equine abortion virus (EHV-1) characterized in that the virus is the EHV1 Ts-mutant deposited at the ECACC under Accession No. V99061001, or progeny thereof.

2. Pharmaceutical composition comprising a temperature-sensitive mutant according to claim 1 and a pharmaceutical acceptable vehicle or carrier.

3. Vaccine for the prevention and/or treatment of EHV-1 infections in equidae comprising a temperature-sensitive mutant virus according to claim 1 and a pharmaceutically acceptable carrier or diluent.

4. Use of a temperature sensitive mutant according to claim 1 in the manufacture of a vaccine for the prevention and/or treatment of EHV-1 infections.

5. A method for the immunization of an animal against EHV-1 infection comprising administering to said animal a vaccine according to claim 4.
